(19) United States
(12) Patent Application Publication
(10) Pub. No.: US 2024/0369563 A1
(43) Pub. Date: Nov. 7, 2024

(54) BIOMARKER COMBINATION FOR ASSESSING RISK OF ADENOMA AND COLORECTAL CANCER, AND USE THEREOF

(71) Applicant: HUMAN METABOLOMICS INSTITUTE, INC., SHENZHEN, GUANGDONG (CN)

(72) Inventors: Wei Jia, Shenzhen, Guangdong (CN); Guoxiang Xie, Shenzhen, Guangdong (CN); Kejun Zhou, Shenzhen, Guangdong (CN)

(21) Appl. No.: 18/562,900

(22) PCT Filed: May 23, 2022
(86) PCT No.: PCT/CN2022/094558

§ 371 Date: Nov. 21, 2023

(30) Foreign Application Priority Data
May 21, 2021 (CN) 202110557839.5

(51) Int. Cl. G01N 33/574; G01N 30/02; G01N 30/72; G01N 30/88; G16H 30/40

(52) U.S. Cl. CPC G01N 33/57488; G01N 30/7206; G01N 30/7233; G01N 30/88; G01N 33/57419; G16H 30/40; G01N 2030/025; G01N 2030/027; G01N 2030/8813

(57) ABSTRACT

Disclosed are a biomarker combination for assessing the risk of adenoma and colorectal cancer, and the use thereof, in particular a metabolic marker for assessing the risk of adenoma and colorectal cancer of a subject, and the use thereof in the preparation of a diagnostic product for evaluating the risk of adenoma and colorectal cancer. The diagnostic marker has the characteristics of high sensitivity and specificity, has higher sensitivity and specificity for early colorectal cancer diagnosis, and can be used for the early discovery of colorectal cancer, so as to buy time for patients, allow treatment to be started as soon as possible, and improve the clinical treatment effect.

The present application claims the priority of a Chinese patent application No. 2021105578395 filed on May 21, 2021. The entire contents of the Chinese patent application are incorporated into the present application by reference.

### FIELD OF TECHNOLOGY

The present invention relates to the technical field of biology, in particular to a biomarker combination for assessment of the risk of adenoma and colorectal cancer, and use and a determination method thereof.

### BACKGROUND

Colorectal cancer has high morbidity and mortality while a classic development process of the colorectal cancer is in line with a slow progression pattern involving adenoma, atypical hyperplasia and advanced carcinoma. Due to accumulation of various genetic and epigenetic mutations within a time period with 10 years as a unit, colorectal malignant tumors are developed.

In early and precancerous stages of the colorectal cancer, patients usually have no obvious clinical symptoms, and the colorectal cancer can be completely cut and removed when found in time to achieve a better therapeutic effect. However, most of a high-risk population do not have a screening opportunity, and most of persons are in an advanced stage or even have distant metastasis when having symptoms. Even when standardized surgery and comprehensive treatment are performed, a poor prognostic effect and a high treatment cost are caused. Therefore, early detection of the colorectal cancer and precancerous lesions thereof is crucial to improve the overall survival rate of patients, which is beneficial for reducing the difficulty of treatment of the disease and reducing diagnosis and treatment costs. Population-based mass screening, which can significantly reduce the morbidity of the colorectal cancer, improve the cure rate and reduce the mortality, has become a major means for prevention and control of cancer in countries all over the world.

Screening objects of the colorectal cancer can be divided into two groups: a general-risk population and a sporadic high-risk population of the colorectal cancer, which have different screening methods and emphases. The former one is defined as the population over 40 years old that has no personal or family history of colorectal cancer, no colorectal adenoma, no inflammatory bowel diseases or other diseases, and an average or low cancer risk level. The latter one refers to the population that has a personal or family history of colorectal cancer, a history of intestinal adenoma and inflammatory bowel diseases not treated for a long time and that is comprehensively determined by combining a body mass index, smoking and drinking histories, diet and exercise and other various risk factors. In addition, a special-risk population with hereditary colorectal cancer is also included, including familial adenomatous polyposis, hereditary non-polyposis colorectal cancer and the like. The high-risk population and the special-risk population should accept colonoscopy directly. As a gold standard of diagnosis, the colonoscopy plays an important role in early detection of precancerous lesions and reduction of the risk of colorectal tumors. However, the general-risk population is large in size, and the colonoscopy is an invasive examination that requires bowel preparation and has the risk of complications, so that patients have low compliance. Painless colonoscopy, with a higher cost, is not suitable for use as a mass screening tool. For such natural population, it is necessary to develop an efficient and reliable screening means for colorectal cancer, adenoma and other precancerous lesions that has low invasion and a low cost.

At present, screening means commonly used for the colorectal cancer in clinical practice include anal digital examination, detection with fecal occult blood, detection with blood tumor markers, endoscopy, imaging examination (CT virtual endoscopy and air-barium double contrast radiography) and the like. A fecal occult blood test is a most widely used screening technology for early diagnosis of the colorectal cancer. Due to the advantages of no invasion and a low cost, the fecal occult blood test is easily accepted by patients and mainly used for screening patients who should receive colonoscopy, which is supported by multiple evidence-based medical evidences. However, the method has relatively low sensitivity and specificity. According to an improved occult blood detection method, an anti-human hemoglobin antibody is used as a primary antibody to carry out immunohistochemical detection on feces. Compared with detection of ferroheme by a peroxidase, the method has improved specificity, but the sensitivity is still lower than 50%, and determination of results has certain subjectivity. In addition to the fecal occult blood, fecal DNA detection can also be used for detecting mutated DNA. Thus, the sensitivity is improved. However, the cost is high, and the method cannot be widely used as a screening means. The blood tumor markers, such as a carcinoembryonic antigen (CEA) and a carbohydrate antigen 19-9 (CA19-9) also have low sensitivity, so that asymptomatic patients are easily missed. In addition, the CEA is not a tumor specific antigen, but a tumor associated antigen, and other endodermal derived tumors (such as gastric cancer, lung cancer, breast cancer and pancreatic cancer) and even chronic colitis may cause increase of the CEA, so that the specificity is not high. At present, the blood tumor markers are only used as auxiliary diagnostic indicators and therapeutic monitoring means for the colorectal cancer. The CT virtual endoscopy is a novel non-invasive examination means produced by combination of a computer virtual reality technology and modern medical imaging, which can replace the air-barium double contrast radiography to observe the whole colon. However, the sensitivity of detecting colonic polyps is directly related to the size of the polyps, and a tested population is exposed to radiation, so that the method has a high examination cost and is still not suitable for screening the natural population.

A patent No. CN201611090742.3 provides a kit for early auxiliary diagnosis of colorectal cancer, a use method thereof and a detection system. The kit includes a nucleic acid separation and purification reagent, a DNA sulfite conversion reagent, a KRAS gene mutation detection reagent, a BMP3 and NDRG4 gene methylation detection reagent and a fecal occult blood detection reagent. The nucleic acid separation and purification reagent is used for separating and purifying human DNA from a fecal sample. The DNA sulfite conversion reagent is used for converting a part of the purified human DNA into a sulfite for subsequent detection of methylation of BMP3 and NDRG4 genes. However, similar genetic tests have the problem of difficulty of distinguishing from common colorectal diseases such as colorectal polyps, and the sensitivity and the specificity remain to be investigated. In addition, the cost is high, and large-scale popularization for use in disease screening is difficult.

A patent No. CN201910446079.3 provides a microRNA biomarker for colorectal cancer. The biomarker includes at least one nucleic acid molecule of a code hsa-miR-423-5p, a code hsa-miR-451a, a code hsa-miR-30b-5p, a code hsa-miR-27b-3p, a code hsa-miR-199a-3p, a code hsa-let-7d-3p and a code hsa-miR-423-5p, and all the nucleic acid molecules are used for encoding a microRNA sequence and have nucleotide sequences shown as SEQ ID NO: 1 to SEQ ID NO: 7, respectively. The microRNA biomarker is obtained by screening of a mixed sample, separate validation of a small amount of samples and separate validation of a large amount of samples. However, similar examinations have the problems of high sampling randomness and low sensitivity for disease detection, so that these examinations are difficult for use in disease screening.

In view of the above situations, it is urgent to develop a simpler and non-invasive detection technology with good compliance and high sensitivity and specificity for screening and early diagnosis of adenoma and colorectal cancer, so as to assess the risk of adenoma and colorectal cancer in a subject more conveniently and reliably.

### SUMMARY

Technical problems to be solved by the present invention are to overcome the defects and shortcomings of the prior art, and to provide a diagnostic marker combination for assessment of the risk of adenoma and colorectal cancer in a subject and a determination method thereof.

The present invention further provides use of the diagnostic marker combination in preparation of a diagnostic product and a computer system for assessment of the risk of adenoma and colorectal cancer in a subject.

In the present invention, the “adenoma and colorectal cancer” includes the situation of “adenoma or colorectal cancer”, and that is to say, an assessment or diagnosis process may be carried out for the purpose of diagnosing or excluding the adenoma and the colorectal cancer simultaneously or for the purpose of diagnosing or excluding one of the diseases.

The “risk” refers to the possibility that the subject has the “adenoma and colorectal cancer”. By means of the method of the present invention, the assessment of the risk may be outputted as a probability value expressed in a number, a “yes” or “no” result obtained by comparing with a preset standard value, or similar qualitative statements expressed.

In order to achieve the above purposes, the present invention provides the following technical schemes.

In a first aspect, the present invention provides a diagnostic product for assessment of the risk of adenoma and colorectal cancer in a subject. The diagnostic product for assessment of the risk of adenoma and colorectal cancer in a subject is characterized in that diagnostic indicators of the diagnostic product include one or more of taurocholic acid, fumaric acid, myristic acid, histidine, tyrosine and 3,4-dihydroxycinnamic acid in a biological sample of the subject, and optionally include a combination A or a combination B below or include the combination A and the combination B simultaneously:
combination A: one or more of 3-hydroxyanthranilic acid, guanidinoacetic acid, azelaic acid, suberic acid, phenylpyruvic acid, acetoacetic acid, methylcysteine, α-hydroxyisobutyric acid, N-methylnicotinamide, salicylic acid, phenylacetic acid, homoserine, glycolic acid, 3-hydroxybutyric acid, phenyllactic acid, methylmalonic acid, succinic acid, 2-hydroxy-2-methylbutyric acid, 4-hydroxyproline, hydroxypropionic acid and p-hydroxyphenylacetic acid;
combination B: one or more of lysine, tryptophan, threonine, citrulline, lactic acid, carnosine, 2-hydroxybutyric acid, suberic acid, 3-hydroxybutyric acid, glutamine, pyruvic acid, uridine, succinic acid, citric acid, aconitic acid, isocitric acid, 2-methylcitric acid, indoleacetic acid, guanidinoacetic acid, azelaic acid, tryptamine, 5-hydroxyindoleacetic acid, spermidine, hippuric acid, phenylacetic acid, acetoacetic acid, m-hydroxyphenylpropionic acid, glycine, p-hydroxyphenylacetic acid, 2-aminobutyric acid, β-hydroxybutyric acid, cystine, pantothenic acid, γ-aminobutyric acid, isoleucine, valine, ornithine, glycerol phosphate, aminoxyacetic acid, 4-hydroxy-L-proline, docosahexaenoic acid, phenylalanine, 3,4-dihydroxybutyric acid, 3-methyladipic acid, pseudouridine, serine, homoserine, putrescine, xanthic acid, α-hydroxyglutaric acid, 3-hydroxybenzoic acid, 3-hydroxyisovalerylcarnitine, 3-hydroxyanthranilic acid, β-alanine, palmitoleic acid, cysteine, glutamic acid, uracil, 5-oxyproline, 2-aminobutyric acid, aspartic acid, asparagine, inositol, homocitrulline, oxyglutaric acid, 3,4-dihydroxymandelic acid, 4-hydroxybenzoic acid, hydroxypropionic acid, 3,4-dihydroxycinnamic acid and vanillic acid;
and the biological sample is selected from urine, blood, saliva and feces of the subject.

The diagnostic product is selected from a kit, a medical instrument, a computer system with a diagnostic module, and a diagnostic device.

In a second aspect, the present invention provides a biomarker combination for assessment of the risk of adenoma and colorectal cancer in a subject. Biomarkers are derived from a biological sample of the subject, and the biological sample includes differential metabolites in the biological sample, such as urine, blood, saliva and feces, of the subject. When the blood is used as the sample, whole blood, serum and plasma may be selected. In some specific embodiments, serum derived from peripheral blood may be selected as the biological sample. In some specific embodiments, the urine of the subject is selected as the biological sample in the present invention, so that sampling is more convenient, and the subject has better compliance.

The present invention provides a biomarker combination for assessment of the risk of adenoma and colorectal cancer in a subject. Biomarkers are differential metabolites in a biological sample of the subject, and the biological sample is selected from urine, blood, saliva and feces; and the biomarker combination includes one or more of taurocholic acid, fumaric acid, myristic acid, histidine, tyrosine and 3,4-dihydroxycinnamic acid, and optionally includes a combination A or a combination B below or includes the combination A and the combination B simultaneously:
combination A: one or more of 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, phenylacetic acid, homoserine, glycolic acid, 3-hydroxybutyric acid, phenyllactic acid, methylmalonic acid, succinic acid, 2-hydroxy-2-methylbutyric acid, 4-hydroxyproline, hydroxypropionic acid and p-hydroxyphenylacetic acid;
combination B: one or more of lysine, tryptophan, threonine, citrulline, lactic acid, carnosine, 2-hydroxybutyric acid, suberic acid, 3-hydroxybutyric acid, glutamine, pyruvic acid, uridine, succinic acid, citric acid, aconitic acid, isocitric acid, 2-methylcitric acid, indoleacetic acid, guanidinoacetic acid, azelaic acid, tryptamine, 5-hydroxyindoleacetic acid, spermidine, hippuric acid, phenylacetic acid, acetoacetic acid, m-hydroxyphenylpropionic acid, glycine, p-hydroxyphenylacetic acid, 2-aminobutyric acid, β-hydroxybutyric acid, cystine, pantothenic acid, γ-aminobutyric acid, isoleucine, valine, ornithine, glycerol phosphate, aminoxyacetic acid, 4-hydroxy-L-proline, docosahexaenoic acid, phenylalanine, 3,4-dihydroxybutyric acid, 3-methyladipic acid, pseudouridine, serine, homoserine, putrescine, xanthic acid, α-hydroxyglutaric acid, 3-hydroxybenzoic acid, 3-hydroxyisovalerylcarnitine, 3-hydroxyanthranilic acid, β-alanine, palmitoleic acid, cysteine, glutamic acid, uracil, 5-oxyproline, 2-aminobutyric acid, aspartic acid, asparagine, inositol, homocitrulline, oxyglutaric acid, 3,4-dihydroxymandelic acid, 4-hydroxybenzoic acid, hydroxypropionic acid, 3,4-dihydroxycinnamic acid and vanillic acid.

In some specific embodiments, the biomarker combination of the present invention includes taurocholic acid, fumaric acid, myristic acid, histidine, tyrosine and 3,4-dihydroxycinnamic acid for realizing the purposes of the present invention.

In some specific embodiments, the biomarker combination of the present invention includes a combination of one or more of lysine, tryptophan, threonine, histidine, citrulline, tyrosine, lactic acid, carnosine, 2-hydroxybutyric acid, suberic acid, 3-hydroxybutyric acid, glutamine, pyruvic acid, uridine, succinic acid, citric acid, aconitic acid, isocitric acid, 2-methylcitric acid, indoleacetic acid, guanidinoacetic acid, azelaic acid, tryptamine, 5-hydroxyindoleacetic acid, spermidine, hippuric acid, phenylacetic acid, acetoacetic acid, m-hydroxyphenylpropionic acid, glycine, p-hydroxyphenylacetic acid, 2-aminobutyric acid, myristic acid, β-hydroxybutyric acid, cystine, pantothenic acid, γ-aminobutyric acid, isoleucine, valine, ornithine, glycerol phosphate, aminoxyacetic acid, 4-hydroxy-L-proline, fumaric acid, docosahexaenoic acid, phenylalanine, 3,4-dihydroxybutyric acid, 3-methyladipic acid, pseudouridine, serine, homoserine, putrescine, xanthic acid, α-hydroxyglutaric acid, 3-hydroxybenzoic acid, 3-hydroxyisovalerylcarnitine, 3-hydroxyanthranilic acid, β-alanine, palmitoleic acid, cysteine, glutamic acid, uracil, 5-oxyproline, 2-aminobutyric acid, aspartic acid, asparagine, inositol, homocitrulline, oxyglutaric acid, taurocholic acid, 3,4-dihydroxymandelic acid, 4-hydroxybenzoic acid, hydroxypropionic acid, 3,4-dihydroxycinnamic acid and vanillic acid for realizing the purposes of the present invention.

In some specific embodiments, the biomarker combination includes a combination of one or more of 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, tyrosine, taurocholic acid, phenylacetic acid, homoserine, histidine, glycolic acid, 3-hydroxybutyric acid, phenyllactic acid, methylmalonic acid, succinic acid, 2-hydroxy-2-methylbutyric acid, 4-hydroxyproline, hydroxypropionic acid, fumaric acid, p-hydroxyphenylacetic acid and myristic acid for realizing the purposes of the present invention.

In some specific embodiments, the biomarker combination includes a combination of one or more of 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, tyrosine, taurocholic acid, phenylacetic acid, homoserine, histidine, glycolic acid, 3-hydroxybutyric acid, phenyllactic acid, methylmalonic acid, succinic acid, 2-hydroxy-2-methylbutyric acid, 4-hydroxyproline, hydroxypropionic acid, fumaric acid, p-hydroxyphenylacetic acid and myristic acid for realizing the purposes of the present invention.

In some specific embodiments, the biomarker combination includes taurocholic acid, and optionally, may be further combined with one or more of fumaric acid, myristic acid, histidine, tyrosine, 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, phenylacetic acid, homoserine, glycolic acid, 3-hydroxybutyric acid, phenyllactic acid, methylmalonic acid, succinic acid, 2-hydroxy-2-methylbutyric acid, 4-hydroxyproline, hydroxypropionic acid and p-hydroxyphenylacetic acid for realizing the purposes of the present invention.

In a third aspect, the present invention provides a method for quantitative detection of the biomarker combination. The method includes treating a biological sample of a subject and then subjecting the biomarker combination in the biological sample to quantitative detection by a liquid chromatography tandem mass spectrometry method and/or a gas chromatography tandem mass spectrometry method.

In a fourth aspect, the present invention provides a kit for quantitative detection of the biomarker combination. The kit includes the biomarkers as detection indicators. The kit includes a biomarker standard product and a biomarker extracting agent, the biomarker extracting agent is selected from mixtures of an organic solvent and water, and the organic solvent is selected from one or more of isopropanol, methanol and acetonitrile. In some specific embodiments, where necessary, the kit includes an internal standard.

In some specific embodiments, optionally, when used in gas chromatography-mass spectrometry, the kit may further include a derivatization reagent.

In a fifth aspect, the present invention provides use of the biomarker combination in preparation of a diagnostic product for assessment of the risk of adenoma and colorectal cancer in a subject, where the diagnostic product is used with expression levels of the biomarker combination as assessment indicators.

In some specific embodiments, the diagnostic product is selected from a kit, a diagnostic device and a computer system.

The computer system for assessment of the risk of adenoma and colorectal cancer in a subject may be a program that can be set in an appropriate computer, and may also be a separate or combined computer device. In order to realize the purposes of the present invention, the computer system includes an information acquisition module and a risk assessment module for adenoma and colorectal cancer; the information acquisition module is at least used for performing the following operation: acquiring detection information of a biomarker combination in a subject sample, where the biomarker combination is selected from the biomarker combination described above; and the risk assessment module for adenoma and colorectal cancer is at least used for performing the following operation: assessing whether the subject has adenoma and colorectal cancer or has the disease risk of adenoma and colorectal cancer based on the level of the biomarker combination acquired by the information acquisition module.

In some specific embodiments, the risk assessment module for adenoma and colorectal cancer is at least used for performing the following operations: inputting the level of the biomarker combination acquired by the information acquisition module into a diagnostic model, and assessing whether the subject has adenoma and colorectal cancer or has the disease risk of adenoma and colorectal cancer according to the diagnostic model.

In a sixth aspect, the present invention provides a screening method for biomarkers associated with adenoma and colorectal cancer, as shown in exemplary description of Example 1 of the present invention for detail.

The present invention has the following beneficial technical effects.

In the present invention, a full spectrum analysis test is carried out on metabolites in biological samples of patients with adenoma and colorectal cancer and healthy persons by means of a liquid chromatography-mass spectrometry instrument (LC-QTOFMS) and a gas chromatography-mass spectrometry instrument (GC-TOFMS) in combination with a bioinformatics tool to find differential metabolites, and the differential metabolites are determined as diagnostic markers for the adenoma and colorectal cancer by validation, which can be used for early detection and diagnosis of the adenoma and colorectal cancer and improve a therapeutic effect on the colorectal cancer. Compared with the prior art, the present invention has the advantages that the unique biomarkers and a combination thereof are provided for the first time to serve as detection indicators, the biomarkers for assessment of the risk of adenoma and colorectal cancer have high sensitivity and specificity in diagnosis of the colorectal cancer and also have high sensitivity and specificity in early diagnosis of colorectal adenocarcinoma, which can be used for early detection of the colorectal cancer so as to save time for patients and start therapy as early as possible, and a clinical therapeutic effect is improved.

### BRIEF DESCRIPTION OF THE DRAWINGS

Drawings forming a part of the present invention are used to provide further understanding of the present invention, and schematic embodiments of the present invention and descriptions thereof are used to illustrate the present invention, rather than to form any limitations of the present invention. In the drawings:

FIG. 1A is a distribution diagram of principal component analysis (PCA) scores for distinguishing patients with colorectal cancer (CRC) and normal controls (control), where a P value shows statistical significance of differences between the group and the normal group.

FIG. 1B is a distribution diagram of orthogonal partial least squares-discriminant analysis (OPLS-DA) scores for distinguishing patients with colorectal cancer (CRC) and normal controls (control).

FIG. 1C shows a correlation coefficient of a model permutation test.

FIG. 2A is a volcano plot of differential metabolites of patients with colorectal cancer identified by OPLS-DA compared with controls (VIP>1. |correlation coefficient|>0.3).

FIG. 2B is a volcano plot of metabolites of patients with colorectal cancer and a control group identified by one-dimensional statistical analysis (p<0.05, significantly increased metabolites in CRC (FC>1, red dot) and significantly decreased metabolites in CRC (FC<1, blue dot)).

FIG. 2C is a heat map of differential biomarkers of patients with colorectal cancer and controls (Z score range of −2 to 2).

FIG. 2D is a box plot of representative differential metabolites between patients with colorectal cancer and healthy controls (p<0.05).

FIG. 3 is a receiver operating characteristic (ROC) curve chart of 27 metabolites in urine samples of patients with colorectal cancer in a training set and healthy controls (including 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, tyrosine, taurocholic acid, phenylacetic acid, homoserine, histidine, glycolic acid, 3-hydroxybutyric acid, phenyllactic acid, methylmalonic acid, succinic acid, 2-hydroxy-2-methylbutyric acid, 4-hydroxyproline, hydroxypropionic acid, fumaric acid, p-hydroxyphenylacetic acid and myristic acid).

FIG. 4 is an ROC curve chart of 27 metabolites in urine samples of patients with colorectal cancer in a validation set and healthy controls (including 3-hydroxyanthranilic acid, guanidinoacetic acid, 3,4-dihydroxycinnamic acid, azelaic acid, suberic acid, phenylpyruvic acid, acetoacetic acid, methylcysteine, α-phenylhydroxyisobutyric acid, N-methylnicotinamide, salicylic acid, tyrosine, taurocholic acid, phenylacetic acid, homoserine, histidine, glycolic acid, 3-hydroxybutyric acid, phenyllactic acid, methylmalonic acid, succinic acid, 2-hydroxy-2-methylbutyric acid, 4-hydroxyproline, hydroxypropionic acid, fumaric acid, p-hydroxyphenylacetic acid and myristic acid).

FIG. 5 is an ROC curve chart of 27 metabolites in urine samples of patients with early colorectal cancer (stage I+II) and healthy controls (including 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, tyrosine, taurocholic acid, phenylacetic acid, homoserine, histidine, glycolic acid, 3-hydroxybutyric acid, phenyllactic acid, methylmalonic acid, succinic acid, 2-hydroxy-2-methylbutyric acid, 4-hydroxyproline, hydroxypropionic acid, fumaric acid, p-hydroxyphenylacetic acid and myristic acid).

FIG. 6 is an ROC curve chart for distinguishing patients with colorectal cancer and patients with adenoma (including a metabolite group composed of 27 metabolites: 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, tyrosine, taurocholic acid, phenylacetic acid, homoserine, histidine, glycolic acid, 3-hydroxybutyric acid, phenyllactic acid, methylmalonic acid, succinic acid, 2-hydroxy-2-methylbutyric acid, 4-hydroxyproline, hydroxypropionic acid, fumaric acid, p-hydroxyphenylacetic acid and myristic acid).

FIG. 7 is an ROC curve chart of 27 metabolites in urine samples of patients with adenoma and healthy controls (including 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, tyrosine, taurocholic acid, phenylacetic acid, homoserine, histidine, glycolic acid, 3-hydroxybutyric acid, phenyllactic acid, methylmalonic acid, succinic acid, 2-hydroxy-2-methylbutyric acid, 4-hydroxyproline, hydroxypropionic acid, fumaric acid, p-hydroxyphenylacetic acid and myristic acid).

FIG. 8 is an ROC curve chart of 6 metabolites in urine samples for distinguishing patients with colorectal cancer and healthy controls (including fumaric acid, myristic acid, histidine, tyrosine, 3,4-dihydroxycinnamic acid and taurocholic acid).

FIG. 9 is an ROC curve chart for distinguishing patients with colorectal cancer and patients with adenoma (including a metabolite group composed of 6 metabolites: fumaric acid, myristic acid, histidine, tyrosine, 3,4-dihydroxycinnamic acid and taurocholic acid).

FIG. 10 is an ROC curve chart of 6 metabolites in urine samples of patients with adenoma and healthy controls (including fumaric acid, myristic acid, histidine, tyrosine, 3,4-dihydroxycinnamic acid and taurocholic acid).

### DESCRIPTION OF THE EMBODIMENTS

The technical schemes of the present invention are described in detail below in combination with specific embodiments and drawings of the present invention. Obviously, the specific embodiments described herein are only a part of the embodiments for realizing the technical schemes of the present invention, and should not be understood as the entire embodiments. It is to be understood that the specific embodiments described herein are intended only to explain the present invention, rather than to limit the present invention. On the basis of the embodiments described herein, all other embodiments obtained by persons of ordinary skill in the art without making creative effort under inspiration shall fall within the scope of protection of the present invention.

### Example 1 Discovery and Determination Method of Biomarkers

Herein, the inventor provides, for example, a step of discovering biomarkers for assessment of the risk of adenoma and colorectal cancer, including a screening method for the biomarkers and a quantitative determination method for a screened biomarker combination. The determination method specifically includes the following steps:
step 1: taking biological samples of patients with adenoma and colorectal cancer and healthy persons and performing appropriate pretreatment;
step 2: analyzing and identifying preliminary differential metabolites in the biological samples of the patients with adenoma and colorectal cancer and the healthy persons by a chromatography-mass spectrometry method in combination with a metabolomics analysis method;
step 3: obtaining further differential metabolites based on the selection criteria that a multidimensional OPLS-DA model has a variable important in projection (VIP) value of greater than 1 and a non-parametric test has a P value of less than 0.05; and
step 4: performing validation by using a logistic regression model to obtain differential metabolites.

The biological samples in the step 1 may be selected from urine samples, blood samples and saliva samples of patients with colorectal cancer, patients with benign adenoma and healthy persons. It is understood by persons of ordinary skill in the art that the biological samples of the same type should be selected from the subjects in a test process.

The chromatography-mass spectrometry method in combination with a metabolomics analysis method in the step 2 includes a liquid/gas chromatography-mass spectrometry method in combination with the metabolomics analysis method.

As one of specific embodiments, a gas chromatography-mass spectrometry method is used in the step 2, and a test may be carried out under the following chromatographic conditions: an Rxi-5ms capillary column; a carrier gas: ultrapure helium; a flow rate: 1.0 mL/min; an injection inlet temperature: 260° C.; a transmission line temperature: 260° C.; an ion source temperature: 210°° C.; an injection volume: 1 μL; an injection method: splitless injection; a heating procedure: starting at 80° C. maintaining the temperature for 2 min, then raising the temperature to 220° C. at a heating rate of 10° C./min, raising the temperature to 240° C. at a heating rate of 5° C./min, raising the temperature to 290° C. at a heating rate of 25° C./min, and finally maintaining the temperature at 290° C. for 8 min; an ion source of mass spectrometry: EI source; an electron bombardment energy: 70 eV; and a scanning range of mass spectrometry: m/z, 40-600, a full scanning mode.

As one of specific embodiments, a liquid chromatography-mass spectrometry method is used in the step 2, and a test is carried out under the following chromatographic conditions: an Agilent ZORBAX Eclipse XDB-C18 column (4.6×6 ent, 5 μm) at a temperature of 30° C.; a mobile phase A including water (containing 0.1% of formic acid) and a mobile phase B including acetonitrile (containing 0.1% of formic acid); an elution gradient of the mobile phases: 0-25 min, 1-100% of B; a flow rate: 0.4 mL/min; and an injection volume: 10 μL. Optimal conditions of time-of-flight mass spectrometry include that (1) in a positive ion mode (ES+), a capillary tube has a voltage of 3,500 V. a sprayer is 45 psig, a drying gas has a temperature of 325° C., and a dryer has a flow rate of 11 L/min; and (2) in a negative ion mode (ES−), a capillary tube has a voltage of 3,000 V, and other parameters are consistent with those in the positive ion mode. During spectrum analysis of metabolites, data are collected by plot and centroid in a mass range of 50-1,000 Da.

During the pretreatment of the samples in the step 1, taking urine and serum samples used in a gas chromatography-mass spectrometry test as an example, the pretreatment includes the following steps: taking 50 μL of serum, and adding 10 μL of chlorophenylalanine (0.1 mg/mL, aqueous solution) and 10 μL of heptadecanoic acid (1 mg/mL, alcohol solution) as an internal standard to monitor the sample reproducibility; then, adding 175 μL of a chloroform-methanol mixed solvent (1:3, v/v), and performing vortex oscillation for 30 s; placing the mixed solution in a centrifuge tube at −20° C. for 10 min to promote protein precipitation; then, performing centrifugation at 13,000 rpm for 10 min, taking and adding 200 μL of a supernatant into a high-recovery sample bottle, and performing vacuum drying at room temperature to obtain a sample product; and subjecting the obtained sample product to derivation by a two-step method, adding 50 μL of methoxyamine (15 mg/mL, pyridine solution) first, performing vortex oscillation for 30 s to carry out a reaction at 30° C. for 90 min, then adding 50 μL of BSTFA (containing 1% of TMCS) to carry out a reaction at 70° C. for 60 min, and performing standing, followed by analysis with GC-TOFMS.

During the pretreatment of the samples in the step 1, taking urine and serum samples used in a liquid chromatography-mass spectrometry test as an example, the pretreatment includes the following steps: mixing 50 μl of a serum sample with 200 μl of a methanol-acetonitrile mixture (5:3, v/v) containing chlorophenylalanine (5 μg/mL, aqueous solution), performing vortex oscillation for 2 min, followed by standing for 10 min, then performing centrifugation at 13,000 rpm for 20 min. and taking a supernatant as a sample to be tested.

By means of the determination method of the present invention, variations of the metabolites of the patients with adenoma and colorectal cancer and the healthy persons can be completely and comprehensively reflected, diagnostic markers of the adenoma and colorectal cancer are found, and a favorable technical support is provided for early diagnosis and prognosis of the adenoma and colorectal cancer.

### Example 2 Subjects, Biological Samples and Grouping

Urine samples of clinically confirmed patients with colorectal adenoma, patients with colorectal cancer and healthy persons were collected, where the samples and the collection were approved by the ethics committee of a medical institution. In the collected samples, 220 cases of the patients with colorectal cancer, 20 cases of the patients with adenoma and 180 cases of the healthy person controls were involved. Combined with Example 1, the samples were detected and analyzed by a chromatography-mass spectrometry instrument after treatment, and metabolic spectrum differences between the patients with adenoma, the patients with colorectal cancer and the healthy person controls were visually displayed by establishing a multidimensional statistical model so as to obtain differential metabolites.

Subjects in the present example include patients with colorectal cancer, patients with colorectal adenoma and healthy subjects that are confirmed according to clinical diagnostic indicators. The samples were divided into a training set and a validation set according to the following scheme, and the tested biological samples were midstream morning urine samples of the subjects during fasting.

### (1) Training Set

100 cases of clinical urine samples of patients with colorectal cancer and 100 cases of urine samples of healthy person controls were involved.

### (2) Validation Set

120 cases of clinical urine samples of patients with colorectal cancer, 80 cases of urine samples of healthy person controls and 20 cases of urine samples of patients with colorectal adenoma were involved.

### Example 3 Determination of Urine Samples by a Gas Chromatography-Mass Spectrometry Instrument (GC-TOFMS)

The samples in the training set were determined by a gas chromatography-mass spectrometry instrument.

Pretreatment of the urine samples: 50 μL of urine was added into a 1.5 mL centrifuge tube, and 10 μL of chlorophenylalanine (0.1 mg/mL, aqueous solution) and 10 μL of heptadecanoic acid (1 mg/mL, alcohol solution) were added as an internal standard to monitor the sample reproducibility. Then, 175 μL of a chloroform-methanol mixed solvent (1:3, v/v) was added, and vortex oscillation was performed for 30 s; and the mixed solution was placed in a centrifuge tube at −20°° C. for 10 min to promote protein precipitation. Then, centrifugation was performed at 13,000 rpm for 10 min, 200 μL of a supernatant was taken and added into a high-recovery sample bottle, and vacuum drying was performed at room temperature.

An obtained sample product was subjected to derivation by a two-step method after pumping, 50 μL of methoxyamine (15 mg/mL, pyridine solution) was added first, vortex oscillation was performed for 30 s to carry out a reaction at 30° C. for 90 min, and then 50 μL of BSTFA (containing 1% of TMCS) was added to carry out a reaction at 70° C. for 60 min. A reaction product was subjected to standing at room temperature for 1 h, followed by analysis with GC-TOFMS.

Determination by GC-TOFMS: A Leco Pegasus HT gas chromatography tandem time-of-flight mass spectrometry instrument (LECO, the United States of America) was used; a chromatographic column was: an Rxi-5ms capillary column (filled with 5% of biphenyl and 95% of dimethyl polysiloxane, Restek, Pennsylvania, the United States of America); a carrier gas was: ultrapure helium; a flow rate was: 1.0 mL/min; an injection inlet temperature was: 260° C.; a transmission line temperature was: 260° C.; an ion source temperature was: 210° C.; an injection volume was: 1 μL; an injection method was: splitless injection; and a heating procedure was: starting at 80° C., maintaining the temperature for 2 min, then raising the temperature to 220° C. at a heating rate of 10° C./min, raising the temperature to 240° C. at a heating rate of 5° C./min, raising the temperature to 290° C. at a heating rate of 25° C./min, and finally maintaining the temperature at 290°° C. for 8 min. An ion source of mass spectrometry was: an EI source; an electron bombardment energy was: 70 eV; and a scanning range of mass spectrometry was: m/z, 40-600, a full scanning mode.

Data analysis and processing were performed by using ChromaTOF software (v4.33, LECO, the United States of America).

### Example 4 Determination of Urine Samples by a Liquid Chromatography Tandem Mass Spectrometry Instrument (LC-TQMS)

The samples in the training set were determined by a liquid chromatography-mass spectrometry instrument.

Pretreatment of the urine samples: 50 μl of a serum sample was mixed with 200 μl of a methanol-acetonitrile mixture (5:3, v/v) containing chlorophenylalanine (5 μg/mL, aqueous solution), vortex oscillation was performed for 2 min, followed by standing for 10 min, then centrifugation was performed at 13,000 rpm for 20 min, and a supernatant was taken for analysis with LC-TOFMS.

Determination by LC-QTOFMS: An ultrahigh performance liquid chromatograph (Waters, the United States of America) equipped with a solvent controller, a column oven and a sample controller was used. Mass spectrometry was performed by using a Waters mass spectrometer (Waters, the United States of America) equipped with an electrospray ionization source. A chromatographic column was as follows: ACQUITY UPLC BEH C18 (100 mm×2.1 mm, 1.7 μm) chromatographic column at a temperature of 40° C. A mobile phase A including water (containing 0.1% of formic acid) and a mobile phase B including acetonitrile (containing 30% of isopropanol) were used; an elution gradient of the mobile phases was: 0-20 min, 5-100% of B; and a flow rate of 0.4 mL/min and an injection volume of 5 μL were used. Optimal conditions of tandem mass spectrometry include that (1) in a positive ion mode (ES+), a capillary tube has a voltage of 1,500 V, an ion source has a temperature of 150° C., a drying gas has a temperature of 550° C., and a dryer has a flow rate of 1,000 L/min; and (2) in a negative ion mode (ES−), a capillary tube has a voltage of 2,000 V, and other parameters are consistent with those in the positive ion mode.

Data analysis and processing were performed by using MassLynx software (v4.1, Waters, the United States of America).

### Example 5 Screening of Differential Metabolic Markers

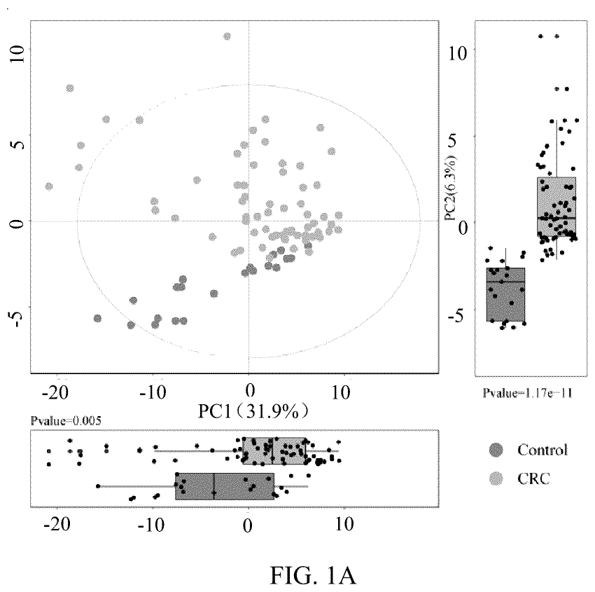

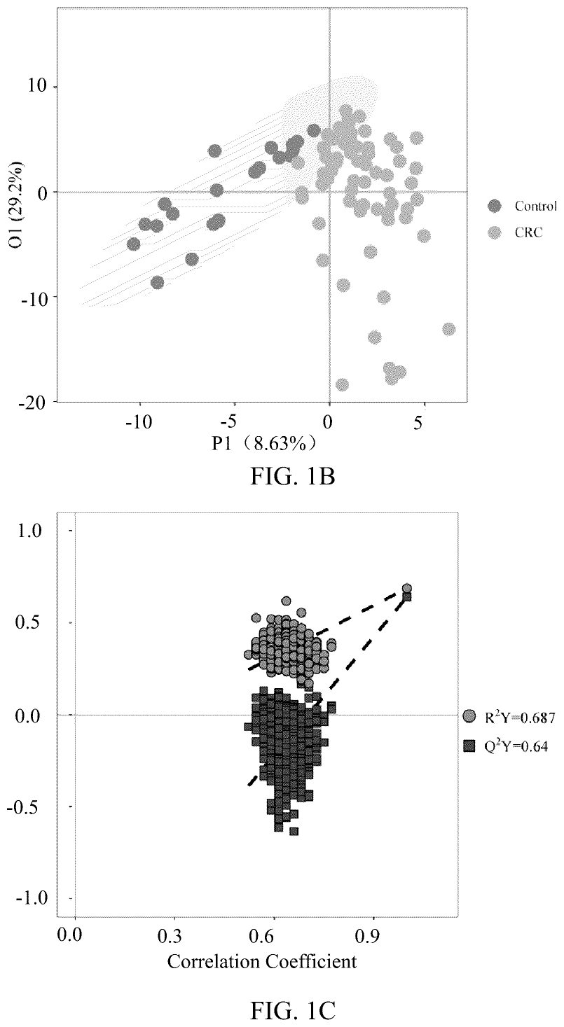

According to Example 3 and Example 4, a full spectrum analysis test was carried out on metabolites by LC-QTOFMS and GC-TOFMS using the samples in the training set. Results are shown in FIG. 1A, FIG. 1B, FIG. 2A, FIG. 2B and FIG. 2C. Based on the selection criteria of a VIP value (VIP>1) provided by multidimensional PCA and OPLS-DA models and a P value (P<0.05) provided by a Mann-Whitney U test, 77 preliminary differential metabolites for distinguishing patients with colorectal cancer and healthy persons as normal controls are obtained from the samples in the training set, including lysine, tryptophan, threonine, histidine, citrulline, tyrosine, lactic acid, carnosine, 2-hydroxybutyric acid, suberic acid, 3-hydroxybutyric acid, glutamine, pyruvic acid, uridine, succinic acid, citric acid, aconitic acid, isocitric acid, 2-methylcitric acid, indoleacetic acid, guanidinoacetic acid, azelaic acid, tryptamine, 5-hydroxyindoleacetic acid, spermidine, hippuric acid, phenylacetic acid, acetoacetic acid, m-hydroxyphenylpropionic acid, glycine, p-hydroxyphenylacetic acid, 2-aminobutyric acid, myristic acid, β-hydroxybutyric acid, cystine, pantothenic acid, γ-aminobutyric acid, isoleucine, valine, ornithine, glycerol phosphate, aminoxyacetic acid, 4-hydroxy-L-proline, fumaric acid, docosahexaenoic acid, phenylalanine, 3,4-dihydroxybutyric acid, 3-methyladipic acid, pseudouridine, serine, homoserine, putrescine, xanthic acid, α-hydroxyglutaric acid, 3-hydroxybenzoic acid, 3-hydroxyisovalerylcarnitine, 3-hydroxyanthranilic acid, β-alanine, palmitoleic acid, cysteine, glutamic acid, uracil, 5-oxyproline, 2-aminobutyric acid, aspartic acid, asparagine, inositol, homocitrulline, oxyglutaric acid, 3,4-dihydroxymandelic acid, 4-hydroxybenzoic acid, hydroxypropionic acid, 3,4-dihydroxycinnamic acid and vanillic acid.

Results are shown in FIG. 2A, FIG. 2B, FIG. 2C and FIG. 2D. FIG. 2A is a volcano plot of differential metabolites of patients with colorectal cancer identified by OPLS-DA compared with controls (VIP>1, |correlation coefficient|>0.3). FIG. 2B is a volcano plot of metabolites of patients with colorectal cancer and a control group identified by one-dimensional statistical analysis (p<0.05, significantly increased metabolites in CRC (FC>1, red dot) and significantly decreased metabolites in CRC (FC<1, blue dot)). FIG. 2C is a heat map of differential biomarkers of patients with colorectal cancer and controls (Z score range of −2 to 2). FIG. 2D is a box plot of representative differential metabolites between patients with colorectal cancer and healthy controls (p<0.05). As can be seen from the results, in the results of a model permutation test (FIG. 1C), an R2Y value is 0.687, a Q2Y value is 0.64, meanwhile, an R2 intercept is less than 0.3, and a Q2 intercept is less than 0.05, proving that the PCA and OPLS-DA models are stably established.

### Example 6 Validation of Biomarkers by Using a Logistic Regression Model

In the present example, a correlation between preliminary differential metabolites and colorectal cancer was studied by exemplarily using a logistic regression model.

The preliminary differential metabolites screened in Example 5 were validated by using a logistic regression model. It is found that 27 metabolites shown in Table 1, including 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, tyrosine, taurocholic acid, phenylacetic acid, homoserine, histidine, glycolic acid, 3-hydroxybutyric acid, phenyllactic acid, methylmalonic acid, succinic acid, 2-hydroxy-2-methylbutyric acid, 4-hydroxyproline, hydroxypropionic acid, fumaric acid, p-hydroxyphenylacetic acid and myristic acid, have an important effect as markers of adenoma and colorectal cancer.

TABLE 1
List of identified differential metabolites

| | | | Change multiple | |
|---|---|---|---|---|
| | | | (patients/healthy | VIP |
| Metabolite | Category | P value | controls) | value |
| 3,4-dihydroxycinnamic | Phenylpropane | 6.21E−06 | 0.42 | 1.85 |
| acid | | | | |
| Phenyllactic acid | Phenylpropanic | 1.49E−03 | 1.52 | 1.19 |
| | acid | | | |
| Azelaic acid | Fatty acid | 4.43E−05 | 2.14 | 1.44 |
| Suberic acid | Fatty acid | 8.43E−03 | 1.20 | 1.94 |
| Myristic acid | Fatty acid | 1.41E−02 | 1.02 | 0.03 |
| Guanidinoacetic acid | Organic acid | 1.16E−04 | 0.63 | 1.57 |
| α-hydroxyisobutyric | Organic acid | 1.26E−04 | 0.49 | 1.87 |
| acid | | | | |
| Glycolic acid | Organic acid | 2.17E−04 | 0.58 | 1.66 |
| 2-hydroxy-2-methylbutyric | Organic acid | 4.09E−04 | 0.61 | 1.69 |
| acid | | | | |
| Hydroxypropionic acid | Organic acid | 1.28E−03 | 0.52 | 1.36 |
| Methylmalonic acid | Organic acid | 1.81E−02 | 0.54 | 1.57 |
| Succinic acid | Organic acid | 2.11E−02 | 0.66 | 1.42 |
| Acetoacetic acid | Organic acid | 2.36E−02 | 1.54 | 2.04 |
| 3-hydroxybutyric acid | Organic acid | 4.19E−02 | 1.45 | 1.92 |
| Fumaric acid | Organic acid | 4.72E−02 | 1.37 | 1.79 |
| 3-hydroxyanthranilic | Benzoic acid | 5.27E−04 | 1.92 | 1.81 |
| acid | | | | |
| Salicylic acid | Benzoic acid | 1.42E−04 | 0.57 | 1.17 |
| Phenylpyruvic acid | Benzoate | 1.88E−02 | 1.35 | 1.14 |
| Phenylacetic acid | Benzoate | 2.36E−02 | 1.51 | 1.11 |
| P-hydroxyphenylacetic | Phenol | 1.78E−02 | 0.74 | 0.98 |
| acid | | | | |
| Taurocholic acid | Bile acid | 2.30E−04 | 0.44 | 1.41 |
| Methylcysteine | Amino acid | 3.12E−04 | 0.51 | 1.31 |
| 4-hydroxyproline | Amino acid | 9.75E−03 | 0.56 | 1.25 |
| Homoserine | Amino acid | 7.14E−04 | 0.58 | 1.44 |
| Tyrosine | Amino acid | 9.48E−04 | 0.60 | 1.88 |
| Histidine | Amino acid | 9.72E−04 | 0.44 | 1.83 |
| N-methylnicotinamide | Pyridine | 1.43E−02 | 1.70 | 1.05 |

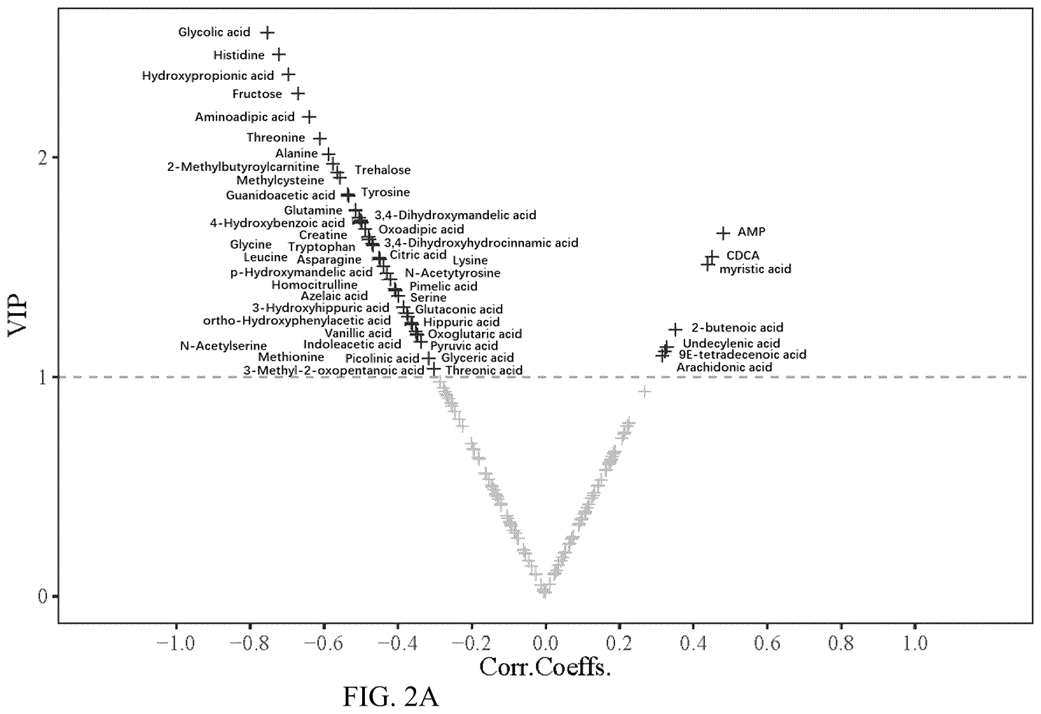

Subsequently, the obtained biomarkers were used in assessment of urine samples of patients with colorectal cancer by using a clinical diagnostic performance curve (namely, ROC curve). Satisfactory results are obtained by using the ROC curve. As for the urine samples in the training set, an area under curve (AUC) value is 0.997, a 95% confidence interval (CIs) is 0.991-1.000 (FIG. 3), the sensitivity is 97%, and the specificity is 100%.

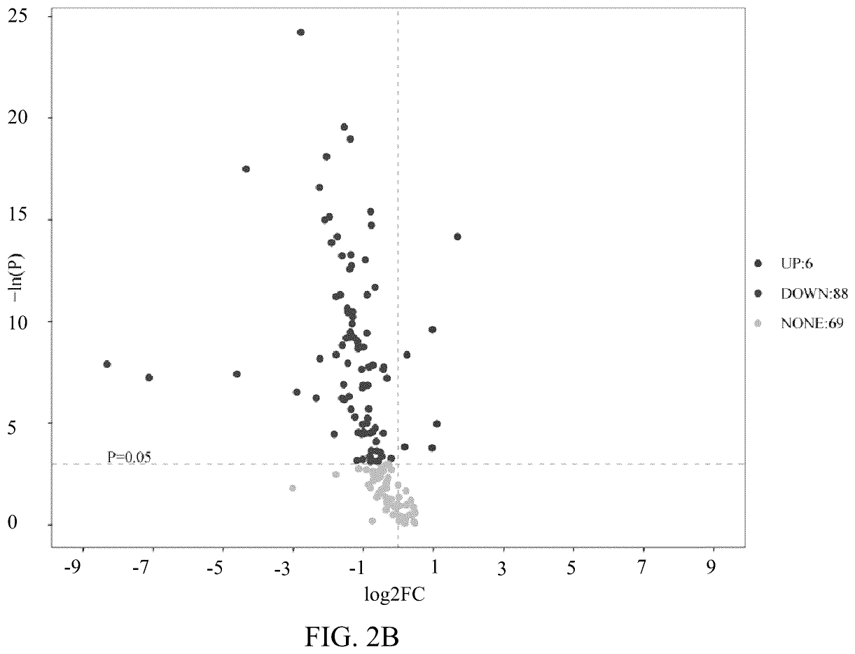

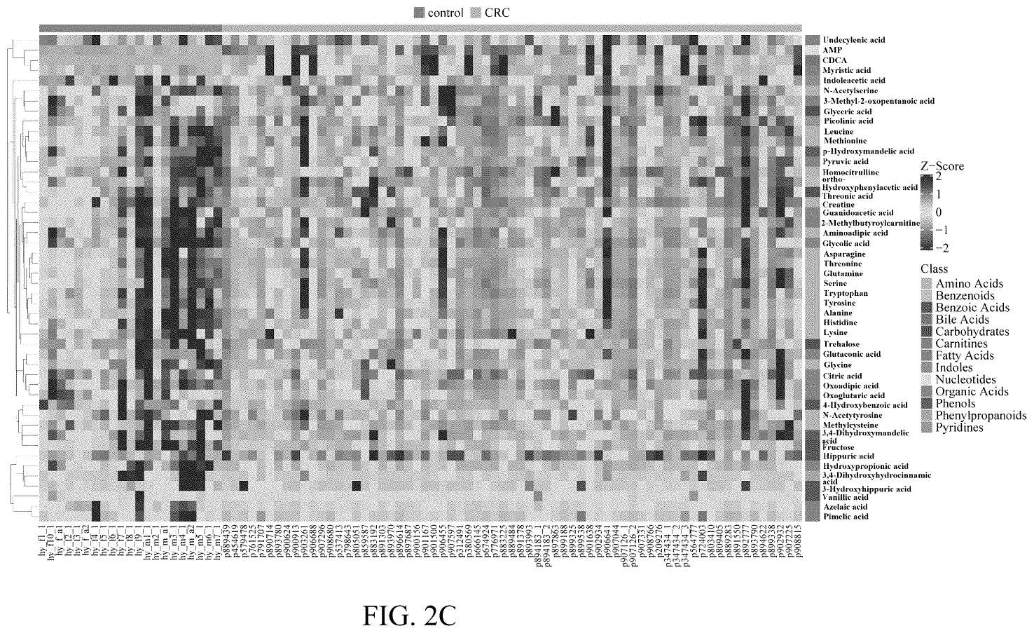

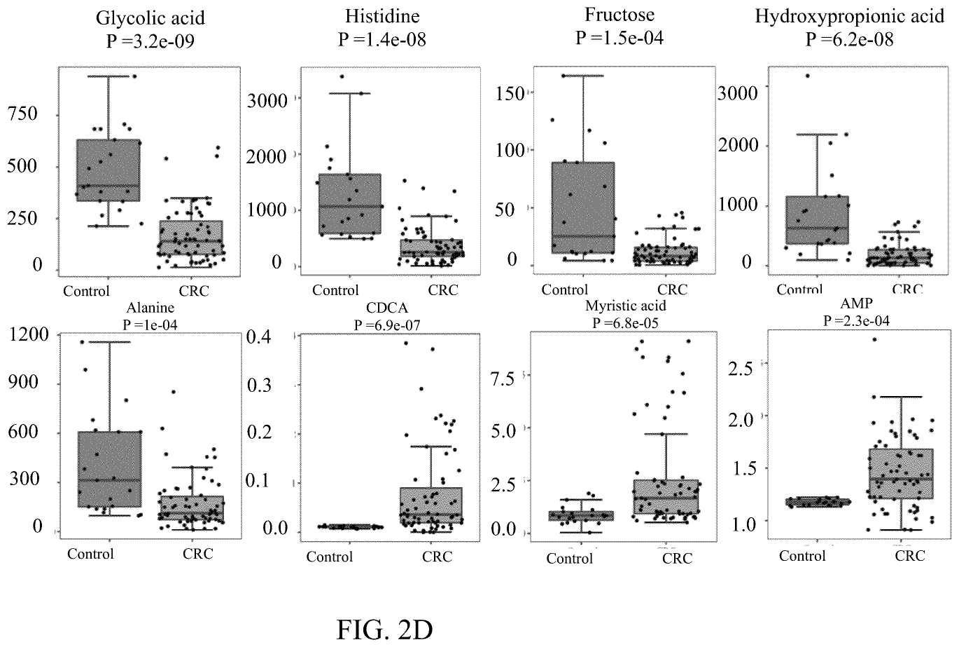

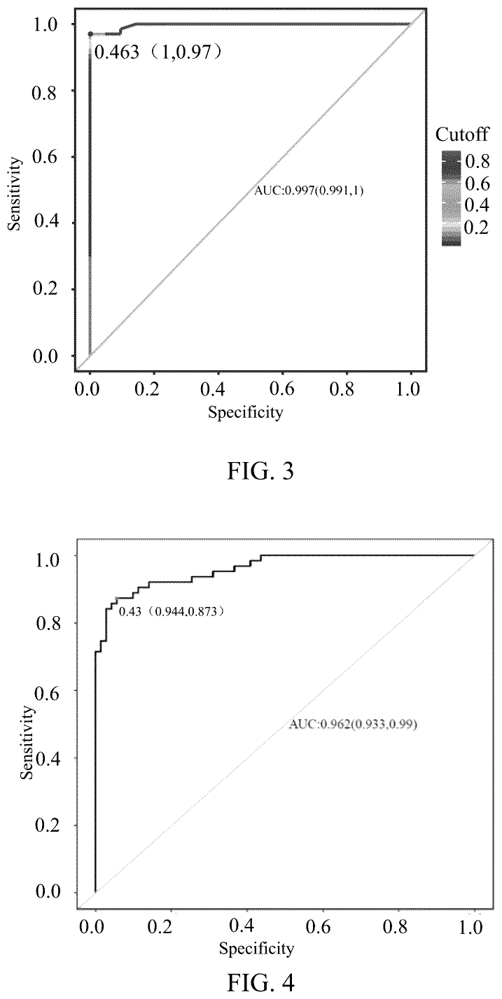

Predicted probability parameters of the 27 metabolites obtained in the training set for the colorectal cancer were validated by using the samples in the validation set. The ROC curve is constructed by using predicted probabilities, where the validation set has an AUC value of 0.962 (95% CIs: 0.933-0.99), and the curve has a sensitivity of 87.5% and a specificity of 94.4% (FIG. 4). However, for the purpose of distinguishing patients with early colorectal cancer (stage I+II) and healthy person controls, the AUC value is 0.974 (95% CIs: 0.949-1.00), and the curve has a sensitivity of 87.9% and a specificity of 100.0% (as shown in FIG. 5). When the 27 metabolites are used for distinguishing 40 patients with colorectal cancer and 20 patients with colorectal adenoma, the AUC value is 0.89, the sensitivity is 78.9%, and the specificity is 87.5% (FIG. 6). For the purpose of distinguishing patients with adenoma and healthy controls, the AUC value is 0.934, the sensitivity is 79.1%, and the specificity is 100.0% (FIG. 7).

As shown in FIG. 3, the 27 biomarkers and a combination thereof in the present example (including 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, tyrosine, taurocholic acid, phenylacetic acid, homoserine, histidine, glycolic acid, 3-hydroxybutyric acid, phenyllactic acid, methylmalonic acid, succinic acid, 2-hydroxy-2-methylbutyric acid, 4-hydroxyproline, hydroxypropionic acid, fumaric acid, p-hydroxyphenylacetic acid and myristic acid) are good markers for early diagnosis of colorectal adenoma and colorectal cancer, which can be used for clinical diagnosis and can improve the early detection rate of adenoma and colorectal cancer, improve the clinical therapeutic effect of adenoma and colorectal cancer, relieve the pain of patients and improve the survival rate of clinical patients.

### Example 7 Establishment of a Biomarker Diagnostic Model

According to Example 5 and Example 6, the 27 differential metabolites that are obtained from the samples in the training set and used for distinguishing patients with adenoma and colorectal cancer and healthy controls include 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, tyrosine, taurocholic acid, phenylacetic acid, homoserine, histidine, glycolic acid, 3-hydroxybutyric acid, phenyllactic acid, methylmalonic acid, succinic acid, 2-hydroxy-2-methylbutyric acid, 4-hydroxyproline, hydroxypropionic acid, fumaric acid, p-hydroxyphenylacetic acid and myristic acid.

For the purpose of establishing an effective clinical diagnostic model, the logistic regression model was further used for optimization and validation. When fumaric acid, myristic acid, histidine, tyrosine, 3,4-dihydroxycinnamic acid and taurocholic acid were used as markers for assessment of the risk of adenoma and colorectal cancer, a score diagnostic model was established by using the logistic regression model according to a difference multiple, magnitude of significance, a concentration value range and other comprehensive factors of the diagnostic markers between patients with adenoma and colorectal cancer and normal controls:

$$model score P = 4.059 * C 1 - 0.019 * C 2 - 0.017 * C 3 - 0.011 * C 4 - 0.056 * C 5 - 0.25 * C 6 + 5.873$$

where C1 refers to the concentration of fumaric acid in a biological sample, C2 refers to the concentration of 3,4-dihydroxycinnamic acid in the biological sample, C3 refers to the concentration of histidine in the biological sample, C4 refers to the concentration of taurocholic acid in the biological sample, C5 refers to the concentration of tyrosine in the biological sample, C6 refers to the concentration of myristic acid in the biological sample, and all the concentrations are used with μM as a unit. According to analysis of a receiver operating characteristic (ROC) curve, a diagnostic threshold for the colorectal cancer is ranged from −0.13 to −0.11, and the optimal diagnostic threshold is −0.12. A diagnostic threshold for the adenoma is ranged from −0.105 to −0.09, and the optimal diagnostic threshold is −0.10. Concentration values of the diagnostic markers were detected for each sample, and then, a score value was calculated according to the diagnostic model and compared with the diagnostic threshold to assess whether a subject has disease or has the disease risk.

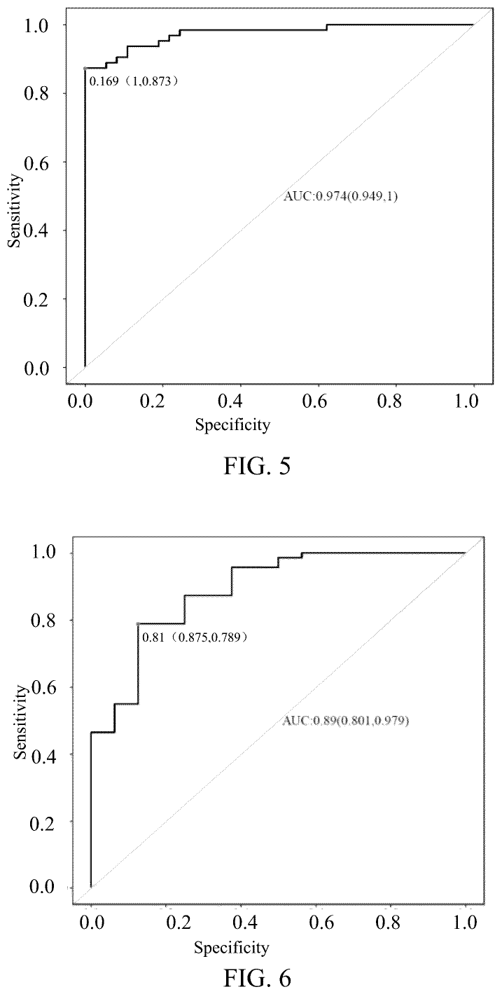

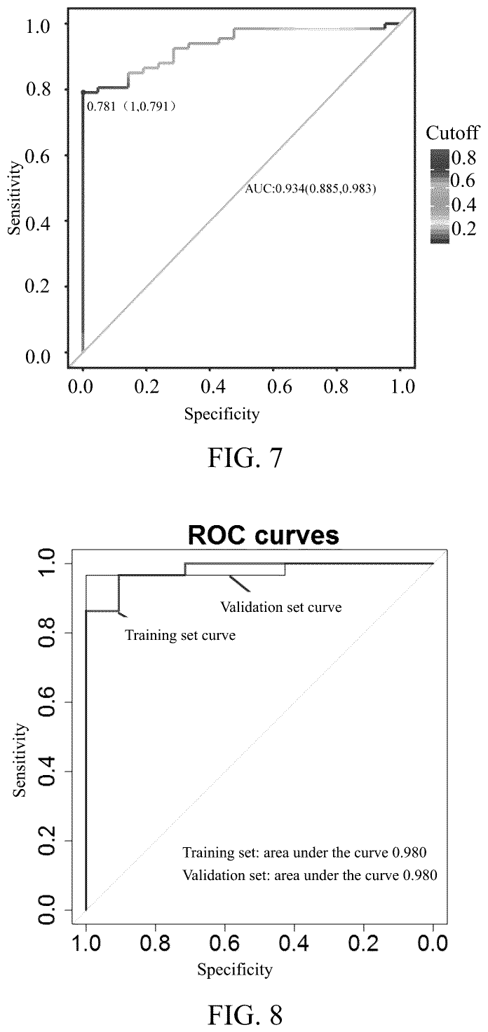

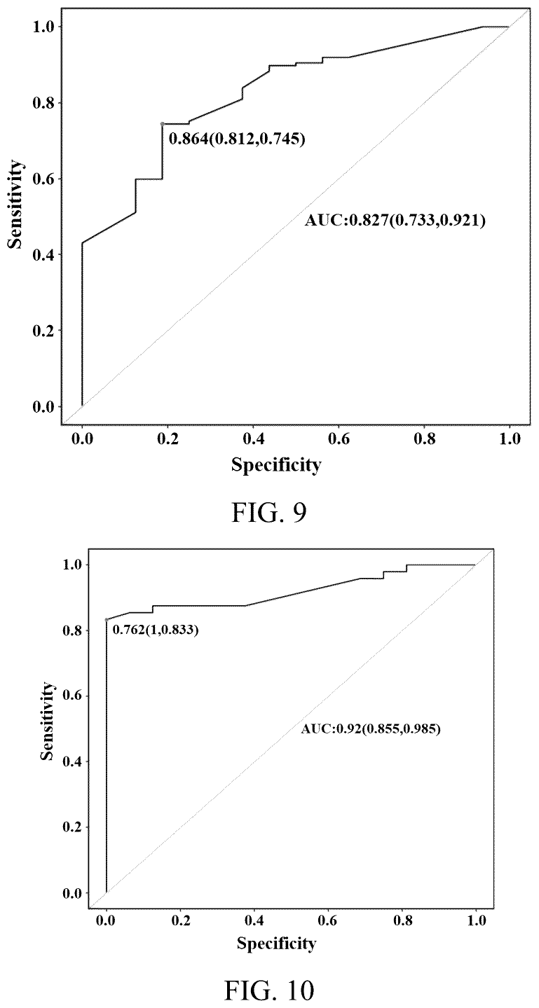

The diagnostic model was used in the training set and the validation set to distinguish patients with colorectal cancer and healthy persons. An ROC curve of the training set has an area under the curve of 0.980, a sensitivity of 96.6% and a specificity of 100%, and an ROC curve of the validation set has an area under the curve of 0.980, a sensitivity of 96.6% and a specificity of 90.5%, as shown in FIG. 8. FIG. 9 is an ROC curve chart of patients with colorectal cancer and patients with adenoma. FIG. 10 is an ROC curve chart of patients with adenoma and healthy controls.

By means of the above method, diagnostic models were established by using the following biomarker combinations:
(1) fumaric acid, myristic acid, histidine, 3,4-dihydroxycinnamic acid and taurocholic acid;
(2) histidine, 3,4-dihydroxycinnamic acid and taurocholic acid;
(3) fumaric acid and taurocholic acid;
(4) taurocholic acid, tyrosine and 3,4-dihydroxycinnamic acid;
(5) fumaric acid, myristic acid, histidine, tyrosine and 3,4-dihydroxycinnamic acid;
(6) fumaric acid, taurocholic acid and histidine;
(7) 3,4-dihydroxycinnamic acid and taurocholic acid;
(8) fumaric acid, taurocholic acid, histidine, 3-hydroxyanthranilic acid, guanidinoacetic acid, hydroxypropionic acid, fumaric acid, p-hydroxyphenylacetic acid and myristic acid;
(9) 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, tyrosine and taurocholic acid; and
(10) 3-hydroxyanthranilic acid, guanidinoacetic acid, 3,4-dihydroxycinnamic acid, salicylic acid, phenyllactic acid, methylmalonic acid, succinic acid, 2-hydroxy-2-methylbutyric acid, 4-hydroxyproline, hydroxypropionic acid, fumaric acid, p-hydroxyphenylacetic acid and myristic acid.

A testing set was validated by using the established diagnostic models. Results show that all the combinations have the abilities to distinguish and diagnose patients with colorectal cancer and healthy persons, patients with adenoma and healthy persons, and patients with colorectal cancer and patients with adenoma, as shown in Table 2.

TABLE 2
Diagnostic abilities of some biomarker combinations

| | AUROC (Area | | |
|---|---|---|---|
| | under ROC | Sensitivity | Specificity |
| Marker combination | curve) | % | % |
| Fumaric acid, myristic acid, | 0.980 | 96.6 | 100 |
| histidine, tyrosine, | | | |
| 3,4-dihydroxycinnamic acid | | | |
| and taurocholic acid | | | |
| Fumaric acid, myristic acid, | 0.92 | 0.876 | 0.817 |
| histidine, 3,4-dihydroxycinnamic | | | |
| acid and taurocholic acid | | | |
| Histidine, 3,4-dihydroxycinnamic | 0.89 | 0.618 | 0.912 |
| acid and taurocholic acid | | | |
| Fumaric acid and taurocholic acid | 0.79 | 0.692 | 0.892 |
| Taurocholic acid, tyrosine and | 0.81 | 0.784 | 0.764 |
| 3,4-dihydroxycinnamic acid | | | |
| Fumaric acid, myristic acid, | 0.87 | 0.698 | 0.881 |
| histidine, tyrosine and | | | |
| 3,4-dihydroxycinnamic acid | | | |
| Fumaric acid, taurocholic | 0.91 | 0.847 | 0.925 |
| acid and histidine | | | |
| 3,4-dihydroxycinnamic acid and | 0.81 | 0.775 | 0.89 |
| taurocholic acid | | | |
| Fumaric acid, taurocholic acid, | 0.85 | 0.866 | 0.814 |
| histidine, 3-hydroxyanthranilic acid, | | | |
| guanidinoacetic acid, | | | |
| hydroxypropionic acid, fumaric acid, | | | |
| p-hydroxyphenylacetic acid and | | | |
| myristic acid | | | |
| 3,4-dihydroxycinnamic acid, azelaic | 0.91 | 0.896 | 0.846 |
| acid, suberic acid, phenylpyruvic | | | |
| acid, acetoacetic acid, | | | |
| methylcysteine, α-hydroxyisobutyric | | | |
| acid, N-methylnicotinamide, | | | |
| salicylic acid, tyrosine and | | | |
| taurocholic acid | | | |
| 3-hydroxyanthranilic acid, | 0.895 | 0.816 | 0.837 |
| guanidinoacetic acid, | | | |
| 3,4-dihydroxycinnamic acid, | | | |
| salicylic acid, phenyllactic acid, | | | |
| methylmalonic acid, succinic acid, | | | |
| 2-hydroxy-2-methylbutyric acid, | | | |
| 4-hydroxyproline, hydroxypropionic | | | |
| acid, fumaric acid, | | | |
| p-hydroxyphenylacetic acid | | | |
| and myristic acid | | | |

### Example 8 Establishment of a Computer System for Detecting Adenomas and Colorectal Cancer

According to Example 1 to Example 7, a computer system for assessment of the risk of adenoma and colorectal cancer in a subject is established in the present example. The system includes an information acquisition module, a module for assessment of the risk of adenoma and colorectal, a sample detection module and a sample pretreatment module.

The information acquisition module is at least used for performing the following operation: acquiring detection information of a biomarker combination in a subject sample, where the biomarker combination is selected from the biomarker combination described above.

The module for assessment of the risk of adenoma and colorectal is at least used for performing the following operation: assessing whether the subject has adenoma and colorectal cancer or has the disease risk of adenoma and colorectal cancer according to the level of the biomarker combination acquired by the information acquisition module, specifically including: inputting the level of the biomarker combination acquired by the information acquisition module into a diagnostic model, and assessing whether the subject has adenoma and colorectal cancer or has the disease risk of adenoma and colorectal cancer according to the diagnostic model.

Establishment of the diagnostic model and an assessment method using the assessment model are referred to the above examples. The computer system in the present example may also optionally include or exclude the sample detection module and/or the sample pretreatment module. The sample detection module is at least used for performing the operation of detecting the level of the biomarker composition in the sample, specifically, at least including the operation of detecting the biomarker composition by liquid chromatography tandem mass spectrometry or by gas chromatography tandem mass spectrometry, and detection conditions are referred to relevant parts of the specification. The sample pretreatment model is at least used for performing the operation of treating the sample before detection and injection, and specific conditions are referred to relevant parts of the specification.

Specific embodiments of the present invention are described in detail above only for the purpose of examples, and the present invention is not limited to the specific embodiments described above. For persons skilled in the art, any equivalent modifications or substitutions of the present invention also fall within the scope of the present invention. Therefore, equivalent changes and modifications made within the spirit and scope of the present invention shall be covered within the scope of the present invention.

1. A diagnostic product for assessment of the risk of adenoma and colorectal cancer in a subject, wherein diagnostic indicators of the diagnostic product comprise one or more of taurocholic acid, fumaric acid, myristic acid, histidine, tyrosine and 3,4-dihydroxycinnamic acid in a biological sample of the subject, and optionally comprise a combination A or a combination B below or comprise the combination A and the combination B simultaneously:
combination A: one or more of 3-hydroxyanthranilic acid, guanidinoacetic acid, azelaic acid, suberic acid, phenylpyruvic acid, acetoacetic acid, methylcysteine, α-hydroxyisobutyric acid, N-methylnicotinamide, salicylic acid, phenylacetic acid, homoserine, glycolic acid, 3-hydroxybutyric acid, phenyllactic acid, methylmalonic acid, succinic acid, 2-hydroxy-2-methylbutyric acid, 4-hydroxyproline, hydroxypropionic acid and p-hydroxyphenylacetic acid;
combination B: one or more of lysine, tryptophan, threonine, citrulline, lactic acid, carnosine, 2-hydroxybutyric acid, suberic acid, 3-hydroxybutyric acid, glutamine, pyruvic acid, uridine, succinic acid, citric acid, aconitic acid, isocitric acid, 2-methylcitric acid, indoleacetic acid, guanidinoacetic acid, azelaic acid, tryptamine, 5-hydroxyindoleacetic acid, spermidine, hippuric acid, phenylacetic acid, acetoacetic acid, m-hydroxyphenylpropionic acid, glycine, p-hydroxyphenylacetic acid, 2-aminobutyric acid, β-hydroxybutyric acid, cystine, pantothenic acid, γ-aminobutyric acid, isoleucine, valine, ornithine, glycerol phosphate, aminoxyacetic acid, 4-hydroxy-L-proline, docosahexaenoic acid, phenylalanine, 3,4-dihydroxybutyric acid, 3-methyladipic acid, pseudouridine, serine, homoserine, putrescine, xanthic acid, α-hydroxyglutaric acid, 3-hydroxybenzoic acid, 3-hydroxyisovalerylcarnitine, 3-hydroxyanthranilic acid, β-alanine, palmitoleic acid, cysteine, glutamic acid, uracil, 5-oxyproline, 2-aminobutyric acid, aspartic acid, asparagine, inositol, homocitrulline, oxyglutaric acid, 3,4-dihydroxymandelic acid, 4-hydroxybenzoic acid, hydroxypropionic acid, 3,4-dihydroxycinnamic acid and vanillic acid;
and the biological sample is selected from urine, blood, saliva and feces of the subject.

2. The diagnostic product for assessment of the risk of adenoma and colorectal cancer in a subject according to claim 1, wherein the diagnostic product is selected from a kit, a medical instrument, a computer system with a diagnostic module, and a diagnostic device.

3. A biomarker combination for assessment of the risk of adenoma and colorectal cancer in a subject, wherein biomarkers are differential metabolites in a biological sample of the subject, and the biological sample is selected from urine, blood, saliva and feces; and the biomarker combination comprises one or more of taurocholic acid, fumaric acid, myristic acid, histidine, tyrosine and 3,4-dihydroxycinnamic acid, and optionally comprises a combination A or a combination B below or comprises the combination A and the combination B simultaneously:
combination A: one or more of 3-hydroxyanthranilic acid, guanidinoacetic acid, 3,4-dihydroxycinnamic acid, azelaic acid, suberic acid, phenylpyruvic acid, acetoacetic acid, methylcysteine, α-hydroxyisobutyric acid, N-methylnicotinamide, salicylic acid, phenylacetic acid, homoserine, glycolic acid, 3-hydroxybutyric acid, phenyllactic acid, methylmalonic acid, succinic acid, 2-hydroxy-2-methylbutyric acid, 4-hydroxyproline, hydroxypropionic acid and p-hydroxyphenylacetic acid;
combination B: one or more of lysine, tryptophan, threonine, citrulline, lactic acid, carnosine, 2-hydroxybutyric acid, suberic acid, 3-hydroxybutyric acid, glutamine, pyruvic acid, uridine, succinic acid, citric acid, aconitic acid, isocitric acid, 2-methylcitric acid, indoleacetic acid, guanidinoacetic acid, azelaic acid, tryptamine, 5-hydroxyindoleacetic acid, spermidine, hippuric acid, phenylacetic acid, acetoacetic acid, m-hydroxyphenylpropionic acid, glycine, p-hydroxyphenylacetic acid, 2-aminobutyric acid, β-hydroxybutyric acid, cystine, pantothenic acid, γ-aminobutyric acid, isoleucine, valine, ornithine, glycerol phosphate, aminoxyacetic acid, 4-hydroxy-L-proline, docosahexaenoic acid, phenylalanine, 3.4-dihydroxybutyric acid, 3-methyladipic acid, pseudouridine, serine, homoserine, putrescine, xanthic acid, α-hydroxyglutaric acid, 3-hydroxybenzoic acid, 3-hydroxyisovalerylcarnitine, 3-hydroxyanthranilic acid, β-alanine, palmitoleic acid, cysteine, glutamic acid, uracil, 5-oxyproline, 2-aminobutyric acid, aspartic acid, asparagine, inositol, homocitrulline, oxyglutaric acid, 3,4-dihydroxymandelic acid, 4-hydroxybenzoic acid, hydroxypropionic acid, 3,4-dihydroxycinnamic acid and vanillic acid.

4. (canceled)

5. (canceled)

6. A kit for quantitative detection of the biomarker combination according to claim 3, wherein the kit comprises a biomarker standard product and a biomarker extracting agent, the biomarker extracting agent is selected from mixtures of an organic solvent and water, and the organic solvent is selected from one or more of isopropanol, methanol and acetonitrile.

7. The kit according to claim 6, wherein the kit comprises a derivatization reagent.

8. A method for quantitative detection of the biomarker combination according to claim 3, wherein the method comprises treating a biological sample of a subject and then subjecting the biomarker combination in the biological sample to quantitative detection by a chromatography-mass spectrometry method in combination with a metabolomics analysis method, and the chromatography-mass spectrometry method in combination with the metabolomics analysis method comprises a liquid chromatography-mass spectrometry method in combination with the metabolomics analysis method and a gas chromatography-mass spectrometry method in combination with the metabolomics analysis method.

9. A computer system for assessment of the risk of adenoma and colorectal cancer in a subject, wherein the system comprises an information acquisition module and a module for assessment of the risk of adenoma and colorectal cancer in the subject;
the information acquisition module is at least used for performing the following operation: acquiring detection information of a metabolic marker combination in a subject sample, wherein the metabolic marker combination is selected from the biomarker combination according to claim 3;
and the module for assessment of the risk of adenoma and colorectal cancer in the subject is at least used for performing the following operation: assessing whether the subject has adenoma and colorectal cancer or has the disease risk of adenoma and colorectal cancer based on the level of the metabolic marker combination acquired by the information acquisition module.

10. A method for assessing the risk of adenoma and colorectal cancer in a subject in need thereof, wherein the method comprises the step of quantifying the biomarker combination according to claim 3 in a biological sample;
preferably, the biological sample is a treated biological sample from the subject; and/or, the quantification of the biomarker combination is performed by a chromatography-mass spectrometry method in combination with a metabolomics analysis method; more preferably, the chromatography-mass spectrometry method in combination with the metabolomics analysis method comprises a liquid chromatography-mass spectrometry method in combination with the metabolomics analysis method and a gas chromatography-mass spectrometry method in combination with the metabolomics analysis method.
